(19) United States
(12) Patent Application Publication
(10) Pub. No.: US 2015/0150897 A1
(43) Pub. Date: Jun. 4, 2015

(54) METHODS OF TREATING HEPATITIS C VIRUS INFECTION IN SUBJECTS WITH CIRRHOSIS

(71) Applicant: Gilead Pharmasset LLC, Foster City, CA (US)

(72) Inventors: Jill M. Denning, Raleigh, NC (US); John G. McHutchison, Woodside, CA (US); G. Mani Subramanian, Los Altos Hills, CA (US); William T. Symonds, III, San Francisco, CA (US)

(21) Appl. No.: 14/557,156

(22) Filed: Dec. 1, 2014

Related U.S. Application Data
(60) Provisional application No. 61/910,631, filed on Dec. 2, 2013.

(51) Int. Cl. A61K 31/7072; A61K 31/4184; A61K 31/4188; A61K 31/7056

(52) U.S. Cl. CPC A61K 31/7072; A61K 31/7056; A61K 31/4184; A61K 31/4188

(57) ABSTRACT

Methods of treating hepatitis C virus infection in a subject with cirrhosis comprising administering to the subject an effective amount of Compound 1.

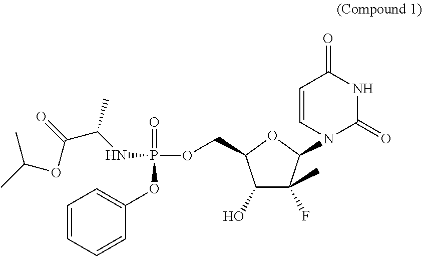 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

### CROSS REFERENCE TO RELATED APPLICATIONS

This application claims the benefits of U.S. Provisional Application No. 61/910,631, filed on Dec. 2, 2013, the entire disclosure of which is incorporated herein by reference.

### FIELD

This application relates to methods of preventing, treating or reducing the risk of hepatitis C virus infection in a subject with cirrhosis.

### BACKGROUND

An estimated 170-180 million people are chronically infected with hepatitis C virus (HCV) worldwide (Ghany et al., “Diagnosis, Management, and Treatment of Hepatitis C: An Update,” Hepatology (2009) 49(4):1335-74). In the United States (US), an estimated 3 million people have chronic HCV infection (Dienstag et al., “American Gastroenterological Association Technical Review on the Management of Hepatitis C,” Gastroenterology (2006) 130(1):231-64). Chronic HCV infection can cause chronic liver disease, cirrhosis, liver failure, hepatocellular carcinoma (HCC) and death. The complications of chronic HCV account for the most common indication for liver transplantation in the US.

The progression to cirrhosis is often clinically silent, and some patients are not known to have HCV until they present with the complications of end-stage liver disease or HCC. Cirrhosis is currently classified as compensated or decompensated based on clinical outcomes. Features of decompensated cirrhosis include the development of ascites, variceal hemorrhage, hepatic encephalopathy, or liver insufficiency (jaundice) (Garcia-Tsao, et al., Hepatology (2010) 51(4):1445-49). In the US, deaths associated with chronic HCV are more likely to be caused from decompensated cirrhosis rather than HCC. Studies have estimated the 3-, 5-, and 10-year survival rates of compensated cirrohisis to be 96%, 91% and 79%, respectively (Chen et al., “The Natural History of Hepatitis C Virus (HCV) Infection,” Int. J. Med. Sci. (2006) 3(2):47-52). The cumulative probability of an episode of clinical decompensation is 5% at 1 year and increases to 30% at 10 years from the diagnosis of cirrhosis (Id.). Once decompensated cirrhosis occurs, the 5-year survival rate falls to 50% (Id.).

While interferon-based therapy may be initiated at a low doses in subjects with decompensated cirrhosis, many subjects who become decompensated have already tried and failed such therapy and/or cannot tolerate the side effects from a course of interferon therapy. Thus, new treatment options for patients with cirrhosis of varying stages are greatly needed.

### SUMMARY

Disclosed herein are methods of treating hepatitis C virus infection in a subject with cirrhosis comprising administering to the subject an effective amount of Compound 1. In some methods, the subject also has portal hypertension. In various methods, Compound 1 may be administered for a duration, wherein the duration ranges from about 1 week to about 48 weeks. Also disclosed are methods of achieving HCV RNA levels below about 25 IU/mL in subject with cirrhosis comprising administering to the subject an effective amount of Compound 1. Also disclosed are methods of maintaining HCV RNA levels below about 25 IU/mL in subject with cirrhosis comprising administering to the subject an effective amount of Compound 1. Such achieving or maintaining HCV RNA levels below about 25 IU/mL in subject with cirrhosis may be at or for a time period after the duration of administration of Compound. This time period may be from about 1 week to about 48 weeks. Also disclosed are methods of treating hepatitis C virus infection, methods of achieving HCV RNA levels below about 25 IU/mL, and methods of maintaining HCV RNA levels below about 25 IU/mL in subject with cirrhosis comprising administering to the subject a 5′-mono-, di- or triphosphate metabolite of Compound 1. In various methods, Compound 1 may be concomitantly administered with at least one additional anti-HCV agent. In some of these methods, this additional anti-HCV agent can be an NS5A inhibitor, such as Compound A or Compound B. In some methods the additional anti-HCV agent is ribavirin.

### DETAILED DESCRIPTION

The abbreviation “ALT” refers to alanine aminotransferase. Increased serum ALT levels can accompany hepatocellular injury or necrosis of striated muscle. In addition, release of ALT from the cytosol may occur secondary to cellular necrosis or as a result of cellular injury with membrane damage. An increased serum ALT level is one indication of hepatic damage.

The abbreviation “AST” refers to aspartate aminotransferase, an enzyme normally found in the blood in low levels. Increased serum AST may result from disease or damage to an organ, including the liver. An increased serum AST level is one indication of hepatic damage.

The term “body mass index” (BMI) refers to the ratio of a subject's weight to the subject's height. BMI is expressed in units of kg/m2 and is calculated as follows:

$$BMI = \frac{weight ( pounds ) \times 703}{{( height in inches )}^{2}} or \frac{weight in kilograms}{{( height in meters )}^{2}}$$

The Child-Pugh (CPT) score, which may also be referred to as the Child-Pugh Turcotte score, is measured on a scale of 5-15 and is used to assess the prognosis of chronic liver disease in subjects with cirrhosis.

“Cirrhosis” refers a CPT score of greater than 5 and includes both compensated cirrhosis and decompensated cirrhosis. A CPT score of 5-6 generally correlates with compensated cirrhosis, and a CPT score of 7-15 generally correlates to decompensated cirrhosis.

“Effective amount” refers to an amount of a compound which, when administered to a subject in need thereof, is sufficient to effect treatment for diseases, conditions, or disorders for which the compound has utility. The effective amount will vary depending on the compound, the disease and its severity, and the age, weight, and other conditions of the subject being or to be treated.

The terms “infected with. HCV,” “HCV infection” and similar terms refer to a documented HCV infection, including acute and chronic infection. HCV infection can be shown, for instance, by a positive anti-HCV antibody test, HCV RNA, or HCV genotyping test.

HCV Viral Load (HCV RNA test, Quantitative) refers to the detection and measurement of the number of viral RNA particles in blood.

Viral genotyping is used to determine the kind, or genotype, of the HCV virus present. There are at least 6 major types of HCV (GT 1-6); the most common in the United States is genotype 1. The abbreviation “GT” refers to genotype.

The abbreviation “IL28B” refers to interleukin 28B, which is one isoform of the interleukin-28 (IL28) cytokine. A single nucleotide polymorphism (SNP) in the IL28B promoter region is identified and is used to predict response to interferon treatment in subjects with HCV.

The abbreviation “IU” refers to international unit, which is a measure of the amount of a substance based on its biological activity or effect.

The abbreviation “LLOQ” refers to the lower limit of quantification. As used herein in reference to HCV RNA measurements, the LLOQ is about 25 IU/mL (as measured by a Roche Cobas® Taqman® Version V2.0 Assay for HCV).

The Milan criteria are applied as a basis for selecting subjects with cirrhosis and hepatocellular carcinoma for liver transplantation. The Milan criteria are defined as follows: (1) a single lesion with a maximum size of 5 cm or (2) up to 3 lesions with the largest not exceeding 3 cm; and (3) no evidence of vascular invasion or extrahepatic manifestations of the cancer as evidenced by imaging (Mazzaferro et al., “Liver Transplantation for the Treatment of Small Hepatocellular Carcinomas in Patients with Cirrhosis,” N. Engl. J. Med. (1996) 334:693-700).

The Model for End-Stage Liver Disease (MELD) score, measured on a scale of 6 to 40, is used for adult liver transplant candidates and is calculated based on bilirubin, international normalized ratio (INR) and creatinine laboratory test results.

“Portal hypertension” refers to a hepatic venous pressure gradient of greater than 5 mmHg.

The terms “prevent” and “preventing” refer to causing the clinical symptoms of the disease not to develop.

The term “subject” refers to a human.

The abbreviation “RVR” refers to rapid virologic response (see, e.g., Poordad et al., Clin. Infect. Dis. (2008) 46:78-84).

A sustained virologic response (SVR) for a subject treated according to one of the methods described herein refers to the subject having HCV RNA less than the lower limit of quantification (LLOQ, e.g., about 25 IU/mL) for a period of time post-treatment as measured in accordance with the assay methodology described herein.

QD means that a dose is administered once a day.

BID means that the dose is administered twice a day. The term “divided BID,” as used herein, means that a total dose amount is divided into two doses administered at different times during the course of a day. The two “divided BID” doses may be equal or different.

Compound 1, (S)-isopropyl2-(((S)-(((2R,3R,4R,5R)-5-(2,4-dioxo-3,4-dihydrophyrimidin-1(2H)-yl)-4-fluoro-3-hydroxy-4-methyltetrahydrofuran-2-yl)methoxy)(phenoxy)phosphoryl)amino)propanoate, is represented by the following chemical structure:

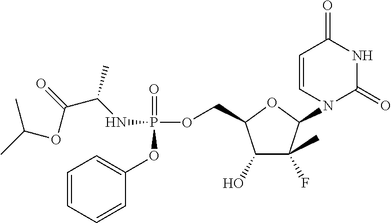 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

(See U.S. Pat. Nos. 7,964,580, 8,642,756, and 8,618,076).

Compound 1 is a nucleotide analog prodrug, the 5′-triphosphate metabolite of which is a potent and selective inhibitor of HCV replication mediated by the HCV NS5B RNA dependent RNA polymerase (Sofia et al., J. Med. Chem. (2010) 53(19):7202-7218).

Compound 1 is converted in vivo to its 5′-mono-, di- and triphosphate metabolites (Id.), represented by the following chemical structures:

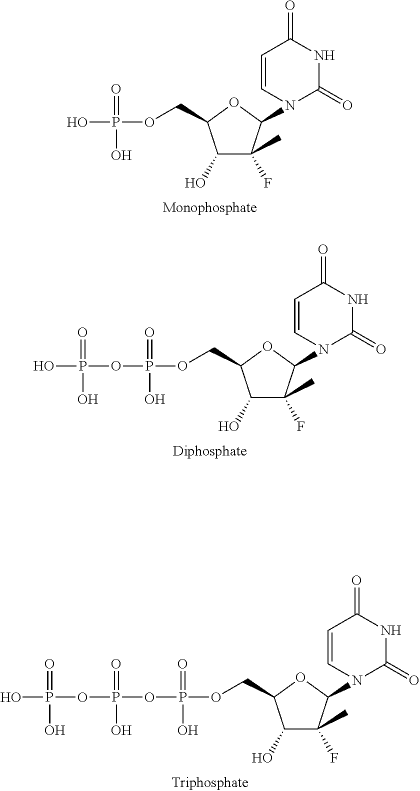 [SMILES: C[C@@]1(F)[C@H](O)[C@@H](COP(=O)(O)O)O[C@H]1n1ccc(=O)[nH]c1=O ; C[C@@]1(F)[C@H](O)[C@@H](COP(=O)(O)OP(=O)(O)O)O[C@H]1n1ccc(=O)[nH]c1=O ; C[C@@]1(F)[C@H](O)[C@@H](COP(=O)(O)OP(=O)(O)OP(=O)(O)O)O[C@H]1n1ccc(=O)[nH]c1=O]

Compound 1 has demonstrated a low incidence of resistance and has been well-tolerated in clinical trials. Compound 1, is also known as “sofosbuvir” or “SOF”.

This application describes various methods of treating hepatitis C virus infection in a subject with cirrhosis. In some methods, the subject may also have portal hypertension.

One method provides for treating hepatitis C virus infection in a subject with cirrhosis comprising administering to the subject an effective amount of Compound 1.

In some methods, the subject has compensated cirrhosis (a CPT score of 5-6) or decompensated cirrhosis (a CPT score of 7-10). Subjects may or may not have esophageal or gastric varices, as observed by endoscopy, within about six months of Compound 1 being administered to the subject. In some methods, the subject has a hepatic venous pressure gradient of greater than 5 mmHg. In some methods, the subject has a hepatic venous pressure gradient of greater than 6 mmHg. In some methods, the subject has a MELD score of 10 or greater.

In various methods, Compound 1 may be administered to the subject for a duration. This duration (which may also be referred to as the duration of administration or duration of the therapy) can be from about 1 week to about 48 weeks, including any and all intervening durations. For example, the duration of therapy can be about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, or about 48 weeks. In some aspects, the duration is 4 weeks. In some aspects, the duration is 8 weeks. In some aspects, the duration is 12 weeks. In other aspects, the duration is 24 weeks. In some aspects, the duration is 48 weeks.

Another method provides for achieving HCV RNA levels below about 25 IU/mL in subject with cirrhosis comprising administering to the subject an effective amount of Compound 1. Another method provides for maintaining HCV RNA levels below about 25 IU/mL in subject with cirrhosis comprising administering to the subject an effective amount of Compound 1. Such achieving or maintaining may be at or for a time period after the duration described above (i.e. the duration of administration of Compound 1, i.e. after the last day on which Compound 1 was administered to the subject). For example, in methods of achieving HCV RNA levels below about 25 IU/mL, HCV RNA levels can be measured at a time period after the duration of administration of Compound 1. Such time period may be from about 1 week to about 48 weeks, for example about 2 weeks, about 4 weeks, about 8 weeks, about 10 weeks, about 12 weeks, or about 24 weeks after the duration of administration of Compound 1. For example, methods provide for achieving an HCV RNA level below about 25 IU/mL in a subject 2 to 24 weeks after the duration of administration of Compound 1. By further example, one method provides for achieving an HCV RNA level below about 25 IU/mL in a subject 2 weeks after the duration of administration of Compound 1. Yet other methods provide for treating hepatitis C virus infection, achieving HCV RNA levels below about 25 IU/mL, and maintaining HCV RNA levels below about 25 IU/mL in subject with cirrhosis comprising administering to the subject a 5′-mono-, di- or triphosphate metabolite of Compound 1.

The effective amount of Compound 1 can be from about 100 mg to about 800 mg per day. For example, the effective amount of Compound 1 can about 200 mg, about 300 mg, about 400 mg, about 500 mg, about 600 mg, about 700 mg, or about 800 mg per day. In some aspects, the effective amount of Compound 1 is 400 mg per day.

In various aspects, the methods comprise administering Compound 1 to the subject QD or BID. For example, certain methods comprise administering to the subject about 400 mg per day of Compound 1 QD.

In various methods, compound 1 can be concomitantly administered with at least one additional anti-HCV agent, for example one additional anti-HCV agent or two or more additional anti-HCV agents. Concomitant administration refers to the administration of two or more agents in any manner in which the pharmacological effects of both agents are manifested in the subject at the same time. Although concomitant administration includes simultaneous administration (e.g. via fixed-dose combinations of two or more agents), it does not require that a single pharmaceutical composition, the same type of formulation, the same dosage form, or even the same route of administration be used for administration of the agents, or that the agents be administered at the same time. Administration of Compound 1 and an additional anti-HCV agent may be concurrent, alternate or any variation thereof, meaning that when the effective amounts of Compound 1 and the additional anti-HCV agent are administered during the same duration, the specific order of administration on a daily basis can be: Compound 1 followed by the additional anti-HCV agent, Compound 1 and the additional anti-HCV agent together, the additional anti-HCV agent followed by Compound I, or any variation thereof. Additionally, it is contemplated that dosage frequencies of Compound 1 and the additional anti-HCV agent may differ. As one non-limiting example, Compound 1 may be administered QD while the additional anti-HCV agent is administered BID.

Examples of additional anti-HCV agents include, without limitation, the following:
A. interferons, for example, pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), or belerofon, IFN alpha-2b XL, rIFN-alpha 2a, consensus IFN alpha, infergen, rebif, pegylated IFN-beta, oral interferon alpha, feron, reaferon, intermax alpha, r-IFN-beta, and infergen+actimmuneribavirin and ribavirin analogs, e.g., rebetol, copegus, VX-497, and viramidine (taribavirin);
B. ribavirin and its analogs, for example, ribavirin (Rebetol, Copegus), and taribavirin (Viramidine);
C. NS5A inhibitors, for example, Compound A.1 (described below), Compound A.2 (described below), Compound A.3 (described below), ABT-267, Compound A.4 (described below), JNJ-47910382, daclatasvir (BMS-790052), ABT-267, Samatasvir, MK-8742, MK-8404, EDP-239, IDX-719, PPI-668, GSK-2336805, ACH-3102, A-831, A-689, AZD-2836 (A-831), AZD-7295 (A-689), and BMS-790052;
D. NS5B polymerase inhibitors, for example, sofosbuvir (GS-7977), Compound A.5 (described below), Compound A.6 (described below), ABT-333, Compound A.7 (described below), ABT-072, Compound A.8 (described below), tegobuvir (GS-9190), GS-9669, TMC647055, ABT-333, ABT-072, setrobuvir (ANA-598), IDX-21437, filibuvir (PF-868554), VX-222, IDX-375, IDX-184, IDX-102, BI-207127, valopicitabine (NM-283), PSI-6130 (R1656), PSI-7851, BCX-4678, nesbuvir (HCV-796), BILB 1941, MK-0608, NM-107, R7128, VCH-759, GSK625433, XTL-2125, VCH-916, JTK-652, MK-3281, VBY-708, A848837, GL59728, A-63890, A-48773, A-48547, BC-2329, BMS-791325, BILB-1941, and ACH-3422;
E. protease (NS3, NS3-NS4) inhibitors, for example, Compound A.9, Compound A.10, Compound A.11, ABT-450, Compound A.12 (described below), simeprevir (TMC-435), boceprevir (SCH-503034), narlaprevir (SCH-900518), vaniprevir (MK-7009), MK-5172, danoprevir (ITMN-191), sovaprevir (ACH-1625), neceprevir (ACH-2684), Telaprevir (VX-950), VX-813, VX-500, faldaprevir (BI-201335), asunaprevir (BMS-650032), BMS-605339, VBY-376, PHX-1766, YH5531, BILN-2065, and BILN-2061;
F. alpha-glucosidase 1 inhibitors, for example, celgosivir (MX-3253), Miglitol, and UT-231B;
G. hepatoprotectants, for example, emericasan (IDN-6556), ME-3738, GS-9450 (LB-84451), silibilin, and MitoQ;
H. TLR-7 agonists, for example, imiquimod, 852A, GS-9524, ANA-773, ANA-975, AZD-8848 (DSP-3025), and SM-360320;
I. cyclophillin inhibitors, for example, DEBIO-025, SCY-635, and NIM811;
J. HCV IRES inhibitors, for example, MCI-067;
K. pharmacokinetic enhancers, for example, BAS-100, SPI-452, PF-4194477, TMC-41629, GS-9350, GS-9585, and roxythromycin;
L. other anti-HCV agents, for example, thymosin alpha 1 (Zadaxin), nitazoxanide (Alinea, NTZ), BIVN-401 (virostat), PYN-17 (altirex), KPE02003002, actilon (CPG-10101), GS-9525, KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, VX-497 (merimepodib) NIM811, benzimidazole derivatives, benzo-1,2,4-thiadiazine derivatives, and phenylalanine derivatives;

Compound A.1 is an inhibitor of the HCV NS5A protein and is represented by the following chemical structure:

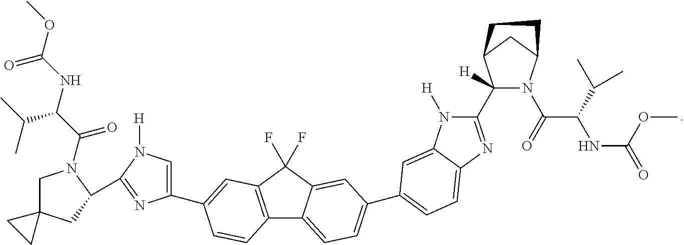

(see, e.g., . U.S. Patent Application Pub. No. 20100310512 A1.).

Compound A.2 is an NS5A inhibitor and is represented by the following chemical structure:

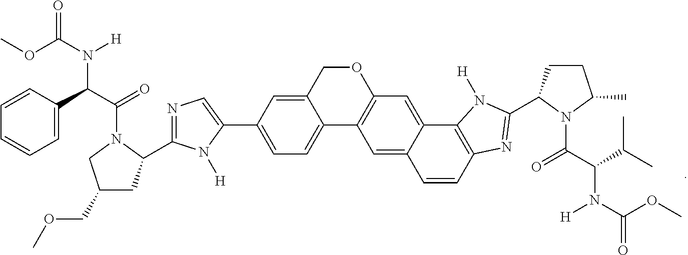

Compound A.3 is an NS5A inhibitor and is represented by the following chemical structure:

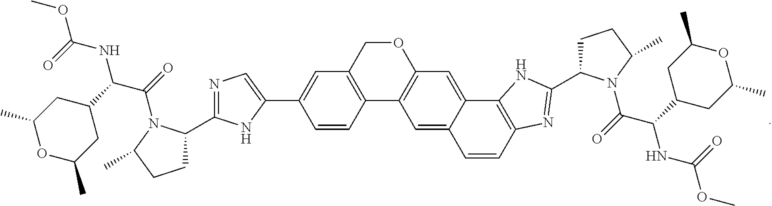

Compound A.4 is an NS5A inhibitor and is represented by the following chemical structure:

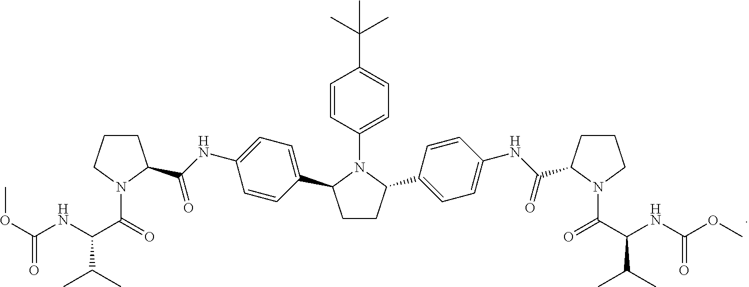 [SMILES: COC(=O)N[C@H](C(=O)N1CCC[C@H]1C(=O)Nc1ccc([C@@H]2CC[C@@H](c3ccc(NC(=O)[C@@H]4CCCN4C(=O)[C@@H](NC(=O)OC)C(C)C)cc3)N2c2ccc(C(C)(C)C)cc2)cc1)C(C)C]

See U.S. Publication No. 2013/0102525 and references therein.

Compound A.5 is an NS5B Thumb II polymerase inhibitor and is represented by the following chemical structure:

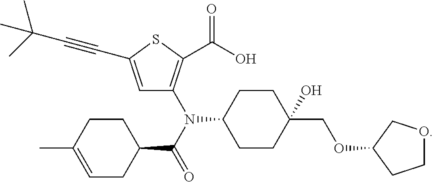 [SMILES: CC1=CC[C@H](C(=O)N(c2cc(C#CC(C)(C)C)sc2C(=O)O)[C@H]2CC[C@](O)(CO[C@H]3CCOC3)CC2)CC1]

Compound A.6 is a nucleotide inhibitor prodrug designed to inhibit replication of viral RNA by the HCV NS5B polymerase, and is represented by the following chemical structure:

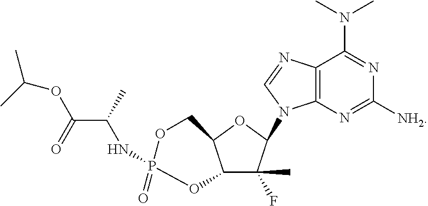 [SMILES: CC(C)OC(=O)[C@H](C)N[P@]1(=O)OC[C@H]2O[C@@H](n3cnc4c(N(C)C)nc(N)nc43)[C@](C)(F)[C@@H]2O1]

Compound A.7 is an HCV polymerase inhibitor and is represented by the following structure:

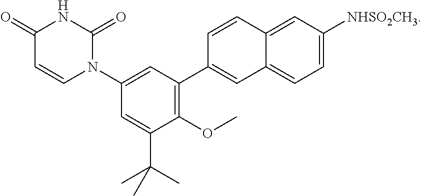

See U.S. Publication No. 2013/0102525 and references therein.

Compound A.8 is an HCV polymerase inhibitor and is represented by the following structure:

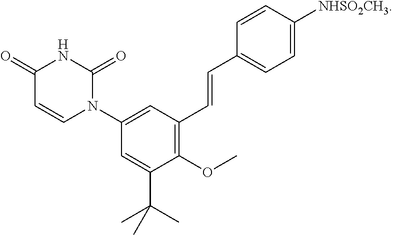

See U.S. Publication No. 2013/0102525 and references therein.

Compound A.9 is an HCV protease inhibitor and is represented by the following chemical structure:

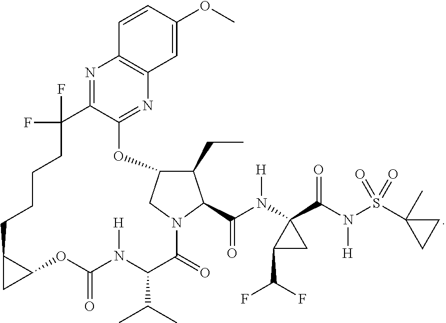 [SMILES: CC[C@@H]1[C@@H]2CN(C(=O)[C@H](C(C)C)NC(=O)O[C@@H]3C[C@H]3CCCCC(F)(F)c3nc4ccc(OC)cc4nc3O2)[C@@H]1C(=O)N[C@]1(C(=O)NS(=O)(=O)C2(C)CC2)C[C@H]1C(F)F]

Compound A.10 is an HCV protease inhibitor and is represented by the following chemical structure:

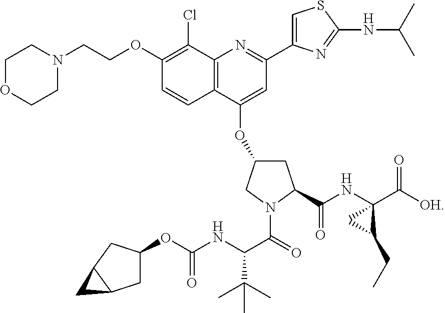

Compound A.11 is an HCV protease inhibitor and is represented by the following chemical structure:

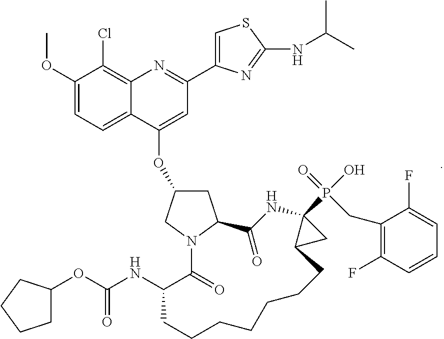 [SMILES: COc1ccc2c(O[C@@H]3C[C@H]4C(=O)N[C@]5(P(=O)(O)Cc6c(F)cccc6F)C[C@H]5CCCCCCC[C@H](NC(=O)OC5CCCC5)C(=O)N4C3)cc(-c3csc(NC(C)C)n3)nc2c1Cl]

Compound A.12 is an HCV protease inhibitor and is represented by the following chemical structure:

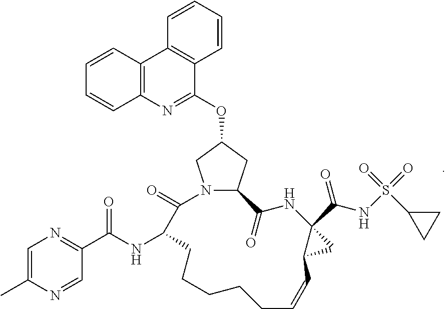 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

See U.S. Publication No. 2013/0102525 and references therein.

In one embodiment, the additional therapeutic agent used in combination with the pharmaceutical compositions as described herein is a HCV NS3 protease inhibitor. Non-limiting examples include the following:

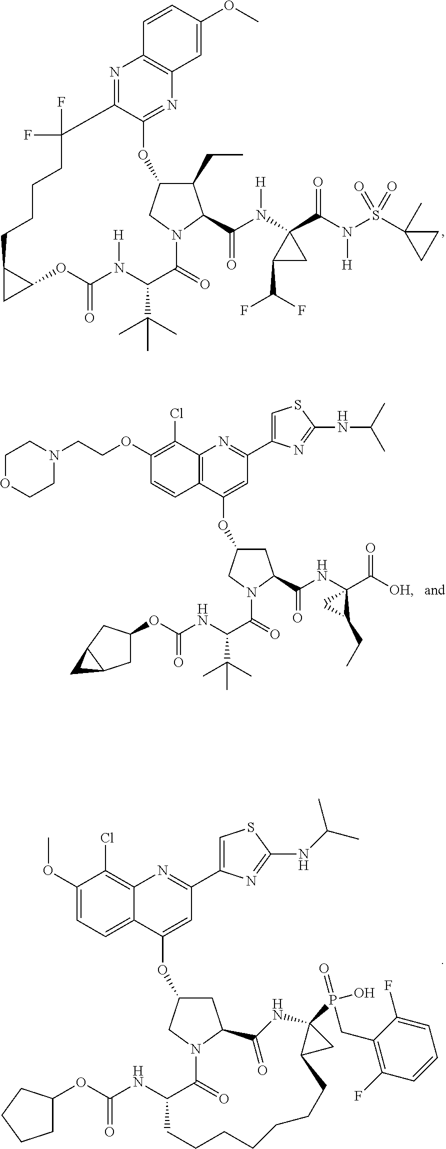

In another embodiment, the additional therapeutic agent used in combination with the pharmaceutical compositions as described herein is a cyclophillin inhibitor, including for example, a cyclophilin inhibitor disclosed in WO2013/185093. Non-limiting examples in addition to those listed above include the following:

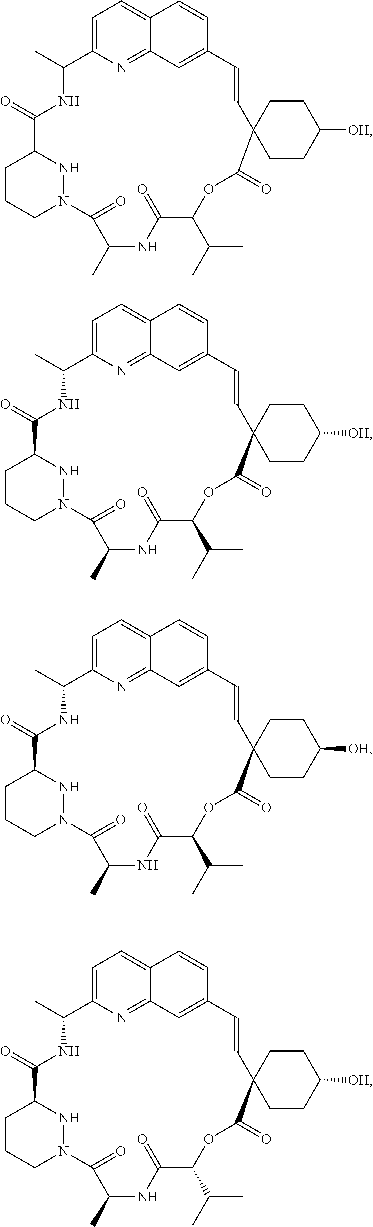

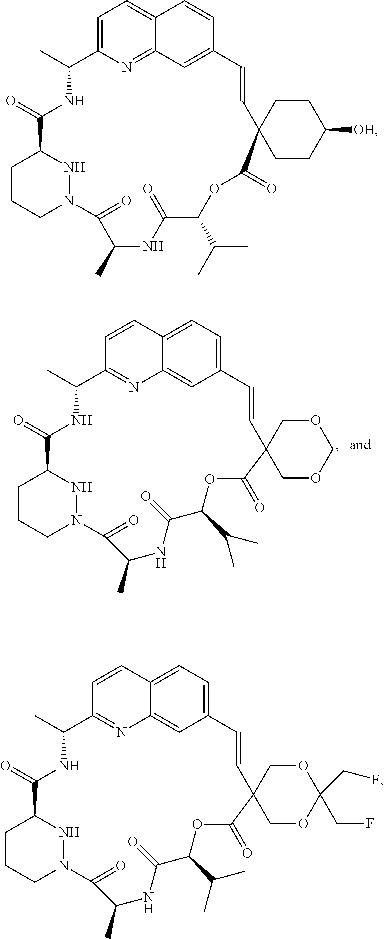

and stereoisomers and mixtures of stereoisomers thereof.

Compound A.1 (also referred to as GS-5885) and compound A.2 (also referred to as GS-9669) have been reported to be useful for the treatment of HCV infection. The results of an open-label, fixed-sequence, cross-over study to evaluate the potential for drug-drug interactions between Compound 1 and GS-5885, GS-9669, or the combination of GS-5885 and GS-9669, have been reported (German et al., “Lack of a Clinically Significant Pharmacokinetic Drug-Drug Interaction Between Sofosbuvir (GS-7977) and GS-5885 or GS-9669 in Healthy Volunteers,” 63rd Annual Meeting of the American Association for the Study of Liver Diseases, Nov. 13, 2012, Boston, Mass., USA). German et al. also reported that increases in Compound 1 exposure from co-administration with GS-5885, GS-9669, and GS-5885+GS-9669 were not clinically significant, and GS-5885 and GS-9669 exposure were unaffected by concurrent administration of Compound 1. This report concluded that no dose adjustment of Compound 1, GS-5885 or GS-9669 is required in combination regimens containing these once-daily direct acting antivirals. In another report, synergy was observed in the HCV replicon assay for the triple combination of Compound 1, GS-5885 and GS-9669 (Hebner et al., “In Vitro Pan-genotypic and. Combination Activity of Sofosbuvir (GS-7977) in Stable Replicon Cell Lines,” 63rd Annual Meeting of the American Association for the Study of Liver Diseases, Nov. 13, 2012, Boston, Mass., USA).

In various methods, Compound A.1 is administered in an amount ranging from about 10 mg/day to about 200 mg/day. For example, the amount of Compound A.1 can be about 30 mg/day, about 45 mg/day, about 60 mg/day, about 90 mg/day, about 120 mg/day, about 135 mg/day, about 150 mg/day, about 180 mg/day. In some methods, Compound A.1 is administered at about 90 mg/day. In various methods, Compound A.2 is administered in an amount ranging from about 50 mg/day to about 800 mg/day. For example, the amount of Compound A.2 can be about 100 mg/day, about 200 mg/day, or about 400 mg/day. In some methods, the amount of Compound A.3 is about 10 mg/day to about 200 mg/day. For example, the amount of Compound A.3 can be about 25 mg/day, about 50 mg/day, about 75 mg/day, or about 100 mg/day.

In certain methods, compound 1 is concomitantly administered with simeprevir. In certain methods, compound 1 is concomitantly administered with MK-8742 or MK-8408. In certain methods, compound 1 is concomitantly administered with MK-5172. In certain methods, compound 1 is concomitantly administered with ABT-450, ABT-267, or ABT-333. In certain methods, compound 1 is concomitantly administered with Viekirat (a combination of ABT-450, ABT-267, and ritonavir). In certain methods, compound 1 is concomitantly administered with daclatasvir.

In certain methods, an effective amount of ribavirin (also an additional anti-HCV agent) can optionally be administered to the subject. In certain methods, no ribavirin is administered. In other methods, the effective amount of ribavirin is from about 100 mg to about 1400 mg per day. In certain methods the effective amount of ribavirin is from about 1000 mg to about 1200 mg per day. In certain methods, the effective amount of ribavirin is based on the subject's body weight. For example, certain methods comprise administering to the subject either about 1000 mg or about 1200 mg per day of ribavirin based on the subject's body weight. Certain methods comprise increasing the daily amount of ribavirin during the course of the duration. For example, certain methods comprise administering to the subject from about 100 mg to about 400 mg per day of ribavirin at the start of the duration, followed by increasing the daily amount of ribavirin up to a maximum amount of from about 200 mg to about 1400 mg per day. Certain methods comprise administering to the subject about 400 mg per day of ribavirin at the start of the duration, followed by increasing the daily amount of ribavirin up to a maximum amount of from about 1000 mg to 1200 mg per day. In certain methods, the daily amount of ribavirin is increased by 200 mg/day/month up to 1000 mg/day or 1200 mg/day, based on the subject's body weight. For example, certain methods comprise administering to the subject about 400 mg per day of ribavirin, followed by increasing the amount of ribavirin by 200 mg/day/month up to a maximum amount of 1000 mg or 1200 mg per day, based on the subject's body weight. Other methods comprise decreasing the daily amount of ribavirin during the course of the duration. For example, certain methods comprise administering to the subject up to about 1200 mg/day at the start of the duration and decreasing the daily amount of ribavirin by 200 mg/day. Other methods comprise administering to the subject about 1400 mg/day at the start of the duration and decreasing the daily amount of ribavirin by 400 mg/day.

In some aspects, ribavirin is administered to the subject QD or BID. For example, in certain methods ribavirin is administered to the subject BID. As a further example, certain methods comprise administering to the subject from about 1000 mg to about 1200 mg per day of ribavirin, divided BID. For example, certain methods comprise administering to the subject about 1000 mg or about 1200 mg per day of ribavirin, based on the subject's body weight, divided BID. Methods may comprise providing to the subject the 5′-mono-, di- and/or triphosphate metabolite of Compound 1 and RBV by administering to the subject Compound 1 and ribavirin according to the foregoing dosage amounts and frequencies.

Some methods comprise administering to the subject from about 100 mg to about 800 mg per day of Compound 1 and from about 100 mg to about 1400 mg per day of ribavirin. For example, in certain aspects the methods comprise administering to the subject about 400 mg per day of Compound 1 and about 400 mg per day, about 600 mg per day, about 800 mg per day, about 1000 mg per day, or about 1200 mg per day of ribavirin. In certain aspects, the methods comprise administering to the subject about 400 mg per day of Compound 1 and about 1000 mg to about 1200 mg per day of ribavirin. In other aspects, the methods comprise administering to the subject about 400 mg per day of Compound 1, and about 1000 mg or about 1200 mg per day of ribavirin, wherein the amount of ribavirin is based on the subject's body weight. In additional aspects, the methods comprise administering to the subject about 400 mg per day of Compound 1 and an initial amount of about 400 mg per day of ribavirin, followed by administering to the subject about 400 mg per day of Compound 1 and increasing the ribavirin amount by 200 mg/day/month up to a maximum amount of about 1000 mg or about 1200 mg per day, depending on the subject's body weight. In further aspects, the methods comprise administering the effective amount of Compound 1 QD and the effective amount of ribavirin divided into multiple amounts (e.g., BID). For example, in certain aspects, the methods comprise administering to the subject about 400 mg per day of Compound 1 QD and about 1000 mg or about 1200 mg per day of ribavirin, based on the subject's body weight, divided BID. In other aspects, the methods comprise administering to the subject about 400 mg per day of Compound 1 QD and an initial amount of about 400 mg per day of ribavirin divided BID, followed by administering to the subject about 400 mg per day of Compound 1 QD and increasing the ribavirin amount by 200 mg/day/month up to a maximum amount of about 1000 mg or about 1200 mg per day, depending on the subject's body weight, divided BID. Methods may comprise providing to the subject the 5′-mono-, di- and/or triphosphate metabolite of Compound 1 and RBV by administering to the subject Compound 1 and ribavirin according to the foregoing dosage amounts and frequencies.

The therapeutic agents and combinations thereof disclosed herein are contemplated to exhibit therapeutic activity when administered in an amount that can depend on the particular case. The variation in amount can depend, for example, on the subject being treated and the active ingredients chosen. A broad range of doses can be applicable. Dosage regimens may be adjusted to provide the optimum therapeutic response. For example, several divided doses may be administered daily, weekly, monthly or other at suitable time intervals or the dose may be proportionally reduced as indicated by the exigencies of the situation. Such dosages are optionally altered depending on a number of variables, not limited to the activity of the one or more active ingredients used, the disease or condition to be treated, the mode of administration, the requirements of the individual subject, the severity of the disease or condition being treated, and the judgment of the practitioner.

As described herein, Compound 1 may be administered (or any of the metabolites thereof described herein may be provided) optionally with at least one additional anti-HCV agent. In the various methods described herein, concomitant administration of these agents is contemplated.

The agents described herein can be formulated into pharmaceutical compositions by combination with appropriate, pharmaceutically acceptable carriers or diluents, and can be formulated into preparations in solid, semi-solid, liquid or gaseous forms, such as tablets, capsules, pills, powders, granules, or other forms.

The 5′-mono-, di- and/or triphosphate metabolites of Compound 1 may be provided to a subject by administering Compound 1 to the subject. For example, the 5′- mono-, di- or triphosphate metabolite of Compound 1 can be provided by administering to the subject about 100 mg to about 800 mg per day, for example 400 mg per day, of Compound 1. The 5′-mono-, di- and/or triphosphate metabolites of Compound 1 may also be provided by other means not involving the administration of Compound 1. For example, it is contemplated that the 5′-mono-, di- and/or triphosphate metabolites of Compound 1 may be provided to a subject by administering compounds other than Compound 1 that are converted to the disclosed 5′-mono-, di- and/or triphosphate metabolites in vivo.

In another aspect, a package or kit is provided comprising Compound 1 and a package insert, a package label, instructions or other labeling relating to the methods of using Compound 1. Certain packages and kits described herein may include statements relating to no significant increases in plasma levels of cyclosporine, sirolimus or tacrolimus when administered concomitantly with Compound 1. Certain packages and kits described herein require no dose adjustment of any cyclosporine, sirolimus or tacrolimus. Certain methods, packages and kits described herein require no warning of a drug-drug interaction between Compound 1 and either cyclosporine, sirolimus or tacrolimus. For example, a package or kit can comprise Compound 1 and a package insert or label describing the administration of Compound 1 for preventing or treating post-liver transplant recurrence of HCV infection and statements indicating no significant increases in plasma levels of cyclosporine, sirolimus or tacrolimus when concomitantly administered with Compound 1.

### EXAMPLES

### Example 1

### Compound 1 and Ribavirin Administered for 48 Weeks to Subjects Infected with Chronic HCV with Cirrhosis and Portal Hypertension

In this open-label study, HCV-infected subjects with compensated and decompensated chronic HCV are administered a combination of Compound 1 and ribavirin. Subjects in the treatment arm (Arm 1) received 48 weeks of 400 mg of Compound 1 QD and 1000 mg or 1200 mg of RBV, divided BID, depending on body weight. Subjects in the untreated observational arm (Arm 2) of the study are rolled into the active arm of the study after 24 weeks of observation.

All subjects met the following criteria, among others: (1) compensated cirrhosis (CTP Score 5-6) or decompensated cirrhosis (CPT Score 7-10) with the presence of esophageal or gastric varices on endoscopy within 6 months prior to or at screening; (2) hepatic venous pressure gradient (HVPG)>6 mmHg; (3) HCV RNA≧103 IU/mL at screening; (4) naïve to all nucleotide/nucleoside treatments for chronic HCV infection.

Subjects' HCV RNA levels are measured at the initial screening visit, baseline (day 1 of treatment), weeks 1, 2, 3, 4 and then every 4 weeks through the end of treatment (24 weeks total), and at 2, 4, 8, 12 and 24 weeks following the last dose of study drug.

A subject's ribavirin drug regimen is modified for the following criteria: (1) hemoglobin<10 g/dL for subjects with no cardiac disease (RBV reduced to 600 mg/day); (2) ≧2 g/dL decrease in hemoglobin during any 4-week period of treatment for subjects with a history of stable cardiac disease (RBV reduced to 600 mg/day); (3) hemoglobin<8.5 g/dL for subjects with no cardiac disease (RBV discontinued); (4) hemoglobin<12 g/dL despite 4 weeks at reduced load for subjects with history of stable cardiac disease (RBV discontinued).

Subjects meeting any of the following criteria are discontinued from therapy: (1) confirmed HCV RNA≧LLoQ after two consecutive HCV RNA<LLoQ; (2) confirmed >1 log 10 increase HCV RNA from nadir; (3) HCV RNA≧LLoQ through 8 weeks of treatment; (4) elevated ALT and/or AST>5× baseline or 5× nadir (unrelated to any interventional procedure); (5) confirmed total bilirubin>3× baseline or 3× nadir with the total bilirubin>5 mg/dL (unless due solely to RBV-induced hemolysis); (6) any grade 2 or greater rash associated with constitutional symptoms; (7) any non-laboratory grade 4 event assessed as related to treatment with COMPOUND 1/RBV; or (8) worsening disease state as evidence by progressing hepatic decompensation and/or progression of Child-Pugh Score (CPT)≧10 or an increase in CPT>2 points from baseline.

HCV RNA is measured using the COBAS® TaqMan® HCV Test, v2.0 for Use with the High Pure System.

HCV genotype and subtype are determined using the Siemens VERSANT® HCV Genotype INNO-LiPA 2.0 Assay.

IL28B genotype is determined by polymerase chain reaction (PCR) amplification of the SNP, rs12979860, with sequence specific forward and reverse primers and allele specific fluorescently labeled TaqMan® MGB probes.

Creatinine clearance is calculated by the Cockcroft-Gault equation using actual body weight (BW) (Cockcroft D W, Gault M H. Prediction of creatinine clearance from serum creatinine. Nephron 1976; 16:31-41):

$$\begin{matrix}Male \text{:} {CL}_{cr} ( mL / min ) = \frac{[ 140 - age ( years ) ] \times BW ( kg )}{72 \times {S}_{cr}} \\ Female \text{:} {CL}_{cr} ( mL / min ) = \frac{[ 140 - age ( years ) ] \times BW ( kg ) \times 0.85}{72 \times {S}_{cr}}\end{matrix}$$

S_{cr}=serum creatinine (mg/dL)

### Example 2

### Interim Report From the HCV-TARGET Real World Experience

Example 2 describes interim observations of a safety and efficacy of all oral HCV therapy in patients with decompensated cirrhosis participating in HCV-TARGET (HCVT), a consortium of academic and community medical centers.

Clinical, adverse events, and virological data were collected throughout treatment and post-treatment follow-up from patients sequentially enrolled between December, 2013 and November, 2014 who were treated with all oral regimens, including Compound 1 (sofosbuvir, or “SOF”). The analyses were restricted to patients with cirrhosis and MELD score of ≧10, who have not undergone liver transplantation in the past. The Safety Cohort consisted of patients who reached an end-treatment (EOT) time point while the Efficacy Cohort were with a known virological outcome (treatment failure or SVR4).

At the time of this interim report, 277 patients with a diagnosis of cirrhosis and a MELD≧10 were started on all oral regimen for HCV treatment, 217 were part of the Safety Cohort, and 156 reached post-treatment follow-up (Efficacy Cohort). Mean age was 59 yrs, 69% male, with MELD score range from 10 to 28 (mean 13.0). 58.5% had failed prior antiviral therapy and 9.7% had failed prior telaprevir or boceprevir triple therapy. Total bilirubin ranged from 0.1-15 (mean 2.1), albumin 1.4-5.0 (mean 3.2), platelets 19K-567K (mean 83K), creatinine 0.4-6 (mean 0.9). Genotype distribution and treatment regimens shown in table. At least one AE was reported in 88% of all patients although most were mild. For patients with available pre/post-treatment bilirubin values, 46/58 (80%), improved, 2/58 (3%) were unchanged, while 10/58 (17%) were worsened. Similarly, among patients with pre/post-treatment albumin values, 33/54 (61%) increased, 7/54 (13%) unchanged, while 14/54 (26%) decreased. 26 patients had baseline MELD and post treatment week 4 data available; 18 had improvement, 5 unchanged, and 3 worsened. SVR 4 rates in those with available data, are shown in Table 1.

TABLE 1

| | | | SOF/SMV/ | |
|---|---|---|---|---|
| | SOF/RBV | SOF/SMV | RBV | All |
| Total Cohort | (n = 120) | (n = 123) | (n = 34) | (n = 277) |
| GT 1 (n/%) | 45 (38%) | 122 (99%) | 32 (94%) | 199 (71.8%) |
| GT 2 | 35 (29%) | 0 (0%) | 0 (0%) | 35 (12.6%) |
| G3 | 37 (31%) | 0 (0%) | 0 (0%) | 37 (13.4%) |
| G4/Other | 2 (2%)/1 (1%) | 0 (0%)/1 (1%) | 2 (6%)/0 (0%) | 4 (1.4%)/2 (1%) |
| MELD | | | | |
| 10-15 | 97 (81%) | 104 (84%) | 29 (85%) | 230 (83%) |
| 16-21 | 20 (17%) | 12 (10%) | 4 (12%) | 36 (13%) |
| >21 | 3 (2%) | 7 (6%) | 1 (3%) | 11 (4%) |
| Discontinued due | 3 (3%) | 5 (4%) | 3 (9%) | 11 (4%) |
| to AE | | | | |
| Discontinued due | 3 (3%) | 0 (0%) | 0 (0%) | 3 (1.1%) |
| to Lack of Efficacy | | | | |
| Died | 0 (0%) | 2 (2%) (liver | 1 (3%) (Cause: | 3 (1.1%) |
| | | failure, | unknown) | |
| | | vascular | | |
| | | shock) | | |
| Virological | (G2 only) | (G1 only) | (G1 only) | |
| response | | | | |
| SVR 4 | 18/24 (75%) | 55/71 (77%) | 13/16 (81%) | |
| Relapse | 3/24 (12%) | 15/71 (21%) | 3/16 (19%) | |
| Breakthrough | 1/24 (4%) | 0/71 (0%) | 0/16 (0%) | |
| Non-response | 1/24 (4%) | 1/71 (1%) | 0/16 (0%) | |
| Loss to F-up | 1/24 (4%) | 0/71 (0%) | 0/16 (0%) | |

SOF: Sofosbuvir;
RBV: ribavirin;
SMV: simeprevir

While the foregoing description describes specific aspects, embodiments, and examples, those with ordinary skill in the art will appreciate that various modifications and alternatives can be developed. Accordingly, the particular aspects, embodiments, and examples described above are meant to be illustrative only, and not to limit the scope of the invention, which is to be given the full breadth of the appended claims, and any and all equivalents thereof.

What is claimed is:

1. A method of treating hepatitis C virus infection in a subject with cirrhosis comprising administering to the subject an effective amount of Compound 1

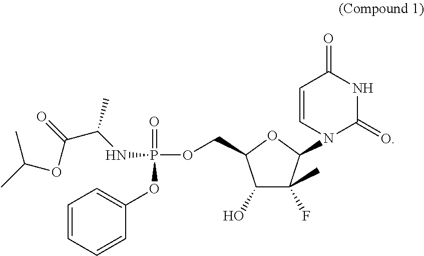 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

2. The method of claim 1, wherein the effective amount of compound 1 is administered to the subject for a duration of about 1 week to about 48 weeks.

3. The method of claim 2, wherein the duration is 48 weeks.

4. The method of claim 1, wherein the effective amount of Compound 1 is from about 100 mg to about 800 mg per day.

5. The method of claim 4, wherein the effective amount of Compound 1 is about 400 mg per day.

6. The method of claim 1, further comprising concomitantly administering to the subject an effective amount of ribavirin.

7. The method of claim 6, wherein the effective amount of ribavirin is from about 100 mg to about 1400 mg per day.

8. The method of claim 7, wherein the effective amount of ribavirin is from about 1000 mg to about 1200 mg per day.

9. The method of claim 6, wherein the effective amount of Compound 1 is about 400 mg per day and the effective amount of ribavirin is from about 1000 mg to about 1200 mg per day.

10. The method of claim 9, wherein the effective amount of Compound 1 and the effective amount of ribavirin are administered to the subject for about 48 weeks.

11. A method of treating hepatitis C virus infection in a subject with cirrhosis comprising providing to the subject the 5′-mono-, di- or triphosphate metabolite of Compound 1.

12. The method of claim 11, wherein the 5′-mono-, di- or triphosphate metabolite of compound 1 is provided to the subject for a duration of about 1 week to about 48 weeks.

13. The method of claim 12, wherein the duration is 48 weeks.

14. The method of claim 11, wherein the 5′-mono-, di- or triphosphate metabolite of Compound 1 is provided by administering to the subject from about 100 mg to about 800 mg per day of Compound 1.

15. The method of claim 14, wherein the 5′-mono-, di- or triphosphate metabolite of Compound 1 is provided by administering to the subject about 400 mg per day of Compound 1.

16. The method of claim 11, further comprising concomitantly administering to the subject an effective amount of ribavirin.

17. The method of claim 16, wherein the effective amount of ribavirin is from about 100 mg to about 1400 mg per day.

18. The method of claim 17, wherein the effective amount of ribavirin is from about 1000 mg to about 1200 mg per day.

19. The method of claim 16, wherein the 5′-mono-, di- or triphosphate metabolite of Compound 1 is provided by administering to the subject about 400 mg per day of Compound 1 and ribavirin is provided by administering to the subject from about 1000 to about 1200 mg per day of ribavirin.

20. The method of claim 16, wherein the 5′-mono-, di- or triphosphate metabolite of Compound 1 and ribavirin are provided to the subject for about 48 weeks.

21. The method of claim 1, further comprising administering at least one additional anti-HCV agent to the subject.

22. The method of claim 21, wherein the at least one additional anti-HCV agent is selected from the group consisting of NS3 protease inhibitors, NS5A inhibitors and NS5B polymerase inhibitors.

23. The method of claim 22, wherein the at least one additional anti-HCV agent is Compound A.1.

24. The method of claim 22, wherein the at least one additional anti-HCV agent is Compound A.2.

25. The method of claim 1, wherein the subject has less than about 25 IU/mL of HCV RNA 2 to 24 weeks after the end of the duration.
